(19) United States
(12) Patent Application Publication
(10) Pub. No.: US 2009/0082660 A1
(43) Pub. Date: Mar. 26, 2009

(54) CLINICAL WORKFLOW FOR TREATMENT OF ATRIAL FIBRULATION BY ABLATION USING 3D VISUALIZATION OF PULMONARY VEIN ANTRUM IN 2D FLUOROSCOPIC IMAGES

(76) Inventors: Norbert Rahn, Forchheim (DE); Stefan Lautenschlager, Forchheim (DE)

Correspondence Address: BRINKS HOFER GILSON & LIONE, P.O. BOX 10395, CHICAGO, IL, 60610, US

(21) Appl. No.: 12/233,230

(22) Filed: Sep. 18, 2008

Related U.S. Application Data
(60) Provisional application No. 60/973,847, filed on Sep. 20, 2007.

(51) Int. Cl. A61B 5/05; A61B 6/00; A61B 18/14

(52) U.S. Cl. 600/411; 378/4; 606/41

(57) ABSTRACT

A system and method of treatment of a patient in a catheterization laboratory is described. A three dimensional (3D) voxel data set of the patient is obtained using a computed tomography device. The data is displayed in a multiplanar slice format, or as a segmented 3D image, and a particular bodily structure identified. The identified structure coordinates are registered, if necessary, with respect to the patient when the patient is positioned for obtaining real-time fluoroscopic images during the treatment, and the bodily structure information is superimposed on the displayed fluoroscopic image. The treatment may be, for example, an electrophysiological (EP) ablation procedure for atrial fibrulation.

This application claims the benefit of priority to U.S. provisional application 60/973,847, filed on Sep. 20, 2007, which is incorporated herein by reference.

### TECHNICAL FIELD

The present application relates to clinical workflow in a catheterization laboratory.

### BACKGROUND

Therapy of atrial fibrillation (AFib) may be performed by minimally invasive electrophysiological (EP) ablation procedures. During such a procedure the pulmonary veins are electrophysiologically isolated from the left atrium by causing ablation lesions in the antrum of the pulmonary veins. These procedures are performed with respect to electrophysiological and morphological structures of the left atrium. A plurality of medical devices are used as part of the procedure for AFib ablations in order to visualize the 3D morphology of the left atrium. Such devices may include: electroanatomical mapping systems (e.g., CARTO from BiosenseWebster, Germany; NavX, from St. Jude Medical) and imaging systems and modalities, which may include different imaging systems and modalities such as C-arm fluoroscopy, intra-procedural 3D C-arm imaging, intracardiac echo, and pre-procedural 3D imaging. These systems are used to visualize an ablation catheter together with the pulmonary vein antrum during the ablation procedure. This enables guidance of the ablation catheter relatively to the left atrial volumetric morphology.

Electroanatomical mapping systems may be used to generate a 3D model of the cardiac chamber and to display the electrophysiological properties of the chamber as colored overlay together with the real-time position and orientation of the ablation catheter during the EP procedure. The 3D model may be inaccurate and the mapping procedure may be cumbersome and time consuming. 3D image data (e.g., CT or MR) may be imported into the mapping systems and registered with the electroanatomical map. However, the required registration procedure might be time consuming and error-prone in some cases.

### SUMMARY

A system for performing a catheterization procedure is described, including a C-arm X-ray device; a catheter system; and a computer. The computer is adapted to store a coordinate data set representing a patient bodily structure, where the data set obtained by analysis of a three-dimensional (3D) voxel data set. A representation of the bodily structure is superimposed on a real-time fluoroscopic image of the patient obtained by the C-arm X-ray device. The voxel data set may be obtained by an imaging device that is different from the C-arm X-ray device, in which case the coordinates of the bodily structure are registered with respect to a fluoroscopic image of the patient.

In an aspect, a method of treatment of a patient is described, the method including: receiving a data set representing a coordinate location of a bodily structure of a patient; obtaining a fluoroscopic image of the patient; if necessary, registering the coordinate location with a coordinate system of the fluoroscopic image; and superimposing the coordinate location of the bodily structure on the fluoroscopic image. In this manner, the relationship of the bodily structure and a treatment device may be visualized on the displayed fluoroscopic image.

In another aspect, a computer program product is described, the product being stored or distributed on a machine readable medium, and having instructions for causing a computer to perform a method of receiving a data set representing a coordinate location of a bodily structure of a patient; and obtaining a fluoroscopic image of the patient. Where the coordinate location data of the bodily structure is obtained by an imaging modality different from that where the patient is positioned for the fluoroscopic images, or the patient has moved since the bodily structure information was determined, the coordinate location of the bodily structure information is registered with respect to a coordinate system of the fluoroscopic image, and the coordinate location information of the bodily structure is superimposed on the fluoroscopic image.

### BRIEF DESCRIPTION OF THE DRAWINGS

FIG. 1 is a block diagram of the platform for performing the workflow of a catheterization procedure;

FIG. 2 shows a four segment display of radiographic data; the upper right and lower left images are MPR (multi-planar reconstruction radiographs) of the left atrium of a patient; the upper left image is a MPR whose orientation is derived from analysis of the other two MPRs and shows the antrum structure substantially in cross-section; and, the lower right image is a segmentation of the 3D data showing the left ventricle;

FIG. 3 is the image group of FIG. 2, highlighting the lines (red) placed by the analyst to orthogonally intersect the lines (blue) which define the centerline of the antrum, so as to select the MPR orientation that is displayed in the upper left segment;

FIG. 4 is the image group of FIG. 2, adding a plurality of points in the antrum cross section image, placed so as to define the outline of the antrum; and

FIG. 5 is the image group of FIG. 4, where the plurality of points of FIG. 4 are displayed in the 3D segmented image of the atrium.

### DETAILED DESCRIPTION

Exemplary embodiments may be better understood with reference to the drawings. Like numbered elements in the same or different drawings perform equivalent functions.

In the interest of clarity, not all the routine features of the examples herein are described. It will of course be appreciated that in the development of any such actual implementation, numerous implementation-specific decisions must be made to achieve a developers' specific goals, such as consideration of system and business related constraints, and that these goals will vary from one implementation to another.

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

When describing a medical intervention technique, the terms “non-invasive,” “minimally invasive,” and “invasive” may be used. Generally, the term non-invasive means the administering of a treatment or medication while not introducing any treatment apparatus into the vascular system or opening a bodily cavity. Included in this definition is the administering of substances such as contrast agents using a needle or port into the vascular system. Minimally invasive means the administering of treatment or medication by introducing a device or apparatus through a small aperture in the skin into the vascular or related bodily structures. Invasive means open surgery.

The combination of hardware and software to accomplish the tasks described herein may be termed a platform. The instructions for implementing processes of the platform may be provided on computer-readable storage media or memories, such as a cache, buffer, RAM, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

The platform may be a catheterization laboratory, and may include ancillary computing and telecommunications devices and networks, or access thereto. Other aspects of the platform may include a remotely located client computer. The client computer may have other functions not related to the platform described herein, and may therefore be shared between users having unrelated functions.

The computer instructions for any processing device may be stored on a removable media device for reading by local or remote systems or processors. In other embodiments, the instructions may be stored in a remote location for transfer through a computer data network, a local area network (LAN) or wide area network (WAN) such as the Internet, by wireless techniques, or over telephone lines. In yet other embodiments, the instructions are stored within a given computer, system, or device.

Where the term “data network”, “web” or “Internet” is used, the intent is to describe an internetworking environment, including both local and wide area networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths.

Communications between the devices, systems and applications may be by the use of either wired or wireless connections. Wireless communication may include, audio, radio, lightwave or other technique not requiring a physical connection between a transmitting device and a compatible receiving device. While the communication may be described as being from a transmitter to a receiver, this does not exclude the reverse path, and a wireless communications device may include both transmitting and receiving functions. A wireless communications connection may include a transceiver implementing a communications protocol such as IEEE 802.11b/g, or the like, such that the transceivers are interoperable.

Where the term “client” is used, a computer executing a program of stored instructions and accepting input from a person, and displaying data, images or the like, in response to such input is meant. Corresponding to the client is another computer, the “server”, that retrieves the data, images, or the like in response to requests received from the client, and transmits the data as information over a communications network. It will be understood by persons of skill in the art that often a computer may act as both a client and a server, and that networks may have intermediate computers, storage devices and the like to provide the functional equivalent of a client and a server interaction protocol. There is no implication herein that any of the functions capable of being performed by a digital computing device, including storage and display devices is restricted to being performed on a specific computer, or in a specific location, even though the description may use such locations or designations for clarity in the examples provided.

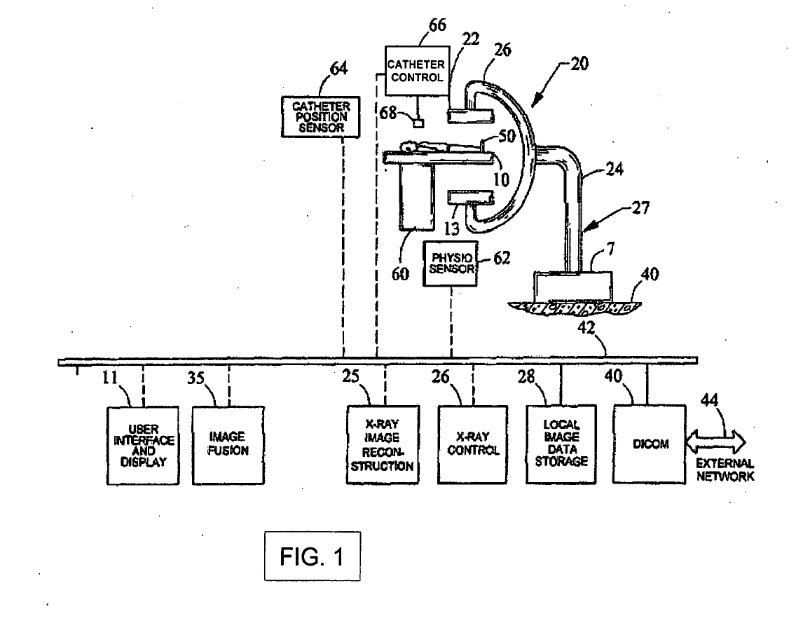

FIG. 1 shows a block diagram of an example of a system for the treatment of an illness by a use of a catheter. In an example, AFib treatment by ablation of an atrium surface of the heart may be performed using minimally invasive techniques. Other embodiments of the system may include more than, or fewer, than all of the devices, or functions, shown in FIG. 1.

The data processing and system control is shown as an example, and many other physical and logical arrangements of components such as computers, signal processors, memories, displays and user interfaces are equally possible to perform the same or similar functions. The particular arrangement shown is convenient for explaining the functionality of the system.

The C-arm X-ray device 20 may comprise a C-arm support 26 to which an X-ray source 22, and an X-ray detector 13 may be mounted so as to face each other about an axis of rotation. The C-arm 26 may be mounted to a robotic device 27 comprising a mounting device 7, and one or more arms 24 which are articulated so as to be capable of positioning the C-arm X-ray device with respect to a patient support apparatus 10. The robotic device 27 may be controlled by a control unit 11, which may send commands causing a motive device (not shown) to move the arms 24. The motive device may be a motor or a hydraulic mechanism. The mounting device may be mounted to a floor 40 as shown, to a ceiling or to a wall, and may be capable of moving in longitudinal and transverse directions with respect to the mounting surface.

The C-arm X-ray device 20 is rotatable in a plurality of planes such that projection X-ray images may be obtained by an X-ray detector 13 positioned on an opposite side of the patient from the X-ray source 22.

The projection X-rays may be obtained as a sequence of images and the images may be reconstructed by any technique of processing for realizing computed tomographic (CT)-like 3D images. 2-D, or real-time fluoroscopic images, may be obtained during the procedure. Depending on the specific procedure, the 3D images may be obtained pre-procedurally or using a different device, which may be a closed CT device, a MR (magnetic resonance imaging) device, or the like, which is not shown.

A patient 50 may be positioned on a patient support apparatus 10. The patient support apparatus 10 may be a stretcher, gurney or the like and may be attached to a robot 60. The patient support apparatus 10 may also be attached to a fixed support or adapted to be removably attached to the robot. Aspects of the patient support apparatus 10 may be manipulable by the robot 60. Additional, different, or fewer components may be provided.

The devices and functions shown are representative, but not inclusive. The individual units, devices, or functions may communicate with each other over cables or in a wireless manner, and the use of dashed lines of different types for some of the connections in FIG. 1 is intended to suggest that alternative means of connectivity may be used.

The C-arm X-ray radiographic device 20 and the associated image processing 25 may produce angiographic and computed tomographic images comparable to, for example, closed-type CT equipment, while permitting more convenient access to the patient for ancillary equipment and treatment procedures. A separate processor 25 may be provided for this purpose, or the function may be combined with other processing functions. The various devices may communicate with a DICOM (Digital Communications in Medicine) system 40 and with external devices over a network interface 44, so as to store and retrieve image and other patient data.

Images reconstructed from the X-ray data may be stored in a non-volatile (persistent) storage device 28 for further use. The X-ray device 20 and the image processing attendant thereto may be controlled by a separate controller 26 or the function may be consolidated with the user interface and display 11. The user interface and display 11 may be a computer workstation that processes image data so as to perform such functions as volume rendering of 3D voxel data sets, production of digitally reconstructed radiographs (DRR), registering of 3D data and 2D data, including voxel data obtained from other imaging modalities, segmenting of the voxel data, and graphical interaction with 3D and 2D data.

Alternatively, some of these functions may be performed on other computing devices, which may be remotely located and communicate with the treatment suite over a network. The display of the images may be on a plurality of displays, of the display may have a plurality of display areas, which may independently display data. An operator may interact with the displays using graphical interaction tools, as is known.

The X-ray images may be obtained with or without various contrast agents that are appropriate to the imaging technology and diagnosis protocol being used.

Additionally, a physiological sensor 62, which may be an electrocardiograph (ECG), a respiration sensor, or the like, may be used to monitor the patient 50 so as to enable selection of images that represent a particular portion of a cardiac or respiratory cycle as a means of minimizing motion artifacts in the images.

The treatment device 66 may be a catheter 68 which is introduced into the body of the patient 50 and guided to the treatment site by images obtained by the C-arm X-ray, or other sensor, such as a catheter position sensor 64. The catheter position sensor may use other than photon radiation, and electromagnetic, magnetic and acoustical position sensors are known.

In order to appropriately direct an ablation catheter to the treatment sites for AFib, visualization of characteristic points of the left atrial morphology in the fluoroscopic images obtained by of the C-arm fluoroscopy system may be performed. The therapeutic intervention may facilitated by interactive identification of the antrum of each of the pulmonary veins (or other characteristic structures of the left atrial morphology) in 3D images by means of image processing software on a 3D workstation 11.

During AFib ablation procedures characteristic, 3D points/lines (especially outlines of the pulmonary vein (PV) antrum) may be identified in a 3D image, which may have been obtained either pre-operatively or intra-operatively, and then transformed for visualization in the real-time 2D fluoroscopy image, taking account of the C-arm orientation. After registering the 3D image with the fluoroscopy images, the characteristic 3D structures, which may be called landmarks, can be overlaid on the 2D fluoroscopic image in order to visually guide the ablation procedure. By this approach may be possible to visualize the PV antrum and the ablation catheter simultaneously in the 2D fluoroscopic image during the ablation procedure. This may permit the catheter guidance to be performed with respect to the 3D morpohology of the appropriate anatomical structure.

In an example of a method using the system of FIG. 1, a method of workflow for performing an AFib procedure may include the following steps: identification of the spatial location of the antrum in a coordinate system of a 3D image data set; registration of 3D images of the patient with 2D fluoroscopic images of the patient; and, displaying the spatial location of the antrium on the 2D fluoroscopic images. In an aspect, the C-arm orientation used to obtain the real-time fluoroscopic images may be changed to obtain a new 2D fluoroscopic image and the spatial location re-displayed on the new 2D image. When the C-arm position is changed, the system may keep track of the orientation, so that the appropriate coordinate transformations may be performed.

The registration of the 2D images with the 3D coordinate system may be performed pre-procedurally or intra-procedurally. In a pre-procedural case, the 3D image data may be acquired by a C-arm X-ray system adapted to produce CT-like images, a computed tomography (CT) device, a magnetic resonance imaging (MR) device, or the like. Where the same imaging device is not used to produce the pre-procedure and intra-procedure image data, or the patient is moved with respect to the imaging device, explicit 2D-3D image registration is needed. Such registration of coordinate systems is a field of study in medical imaging, and a variety of existing techniques are available to perform this function. Others are being developed so as to improve the accuracy and reliability of the registration and to reduce computation time. The registration may also be performed by appropriately transforming the coordinates of the CT scanner into the coordinates of the C-arm X-ray device, so as to locate the patient; for this purpose, the patient may be transported between the two modalities on the patient support device.

In an aspect, the 2D-3D registration may be achieved by performing a 3D acquisition/reconstruction of 3D image information of the heart or of 3D structures next to the heart (e.g., the spine) via the X-ray C-arm system, resulting in intra-procedural 3D image data, and subsequently performing a 3D-3D registration of pre-procedural 3D image data and the intra-procedural image data.

In the intra-procedural case, 3D image data, such as may be obtained by the C-arm X-ray device may be used. In such a circumstance, so long as the patient does not move between the time of 3D image acquisition and performance of the ablation procedure, explicit 2D-3D coordinate registration may not be needed. But, in either the pre-procedural or intra-procedural 3D data acquisition, if the patient moves, or is moved, the registration of 2D and 3D coordinate systems may be explicitly performed, unless the relationship of the old an the new coordinate systems is known.

The spatial location of a bodily structure, such as the antrum line may be identified so as to aid in the performance of the procedure. This may be done by the identification of landmark points of the organ which may be important for the guiding of a catheter, such as the ablation catheter during an AFib procedure. Such landmarks may be identified in the cardiac 3D image which, if necessary, is registered with respect to the X-ray C-arm system and then visualized in the real-time 2D fluoroscopic images during the procedure.

The landmarks used may be, for example, 3D polygon lines or 3D points representing the planned ablation lesion in the pulmonary vein (PV) antrum; 3D points representing the middle of the pulmonary vein antrum; or, 3D polygon lines representing the planned ablation lesions.

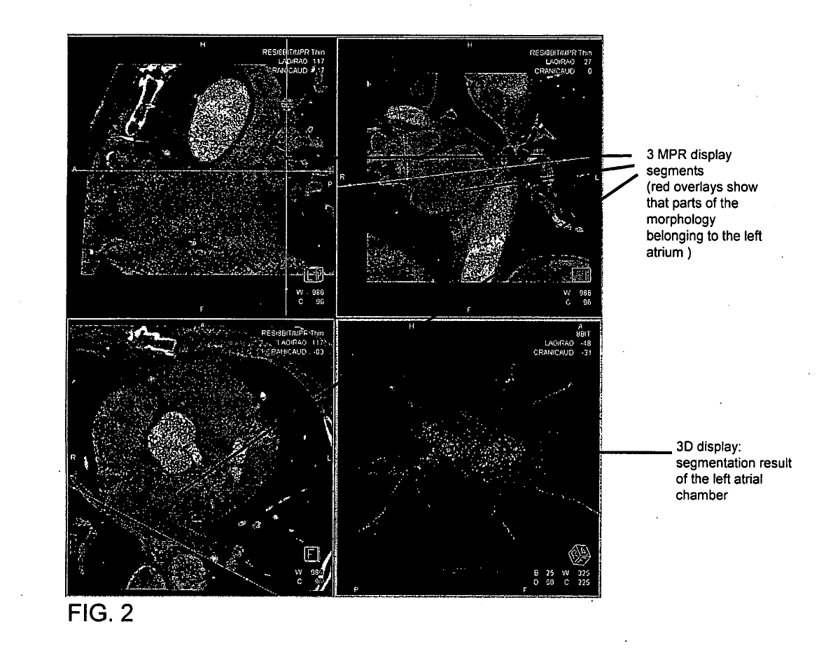

As an example, a procedure for identifying landmarks useful in performing ablation lesions in the pulmonary vein antrum is described. The three-dimensional intra-procedural or pre-procedural image data are displayed on a 3D workstation in a 2×2 display layout such as shown in FIG. 2, where 3 of the display segments are representing 3 multi-planar reconstructions (MPR) and the fourth segment represents the 3D morphology of the chamber to be ablated. MPRs are digitally reconstructed radiographs (DRR), which are 2D images. Each reconstruction of a MPR is equivalent to a slice image of a volumetric data set at an arbitrarily selected orientation.

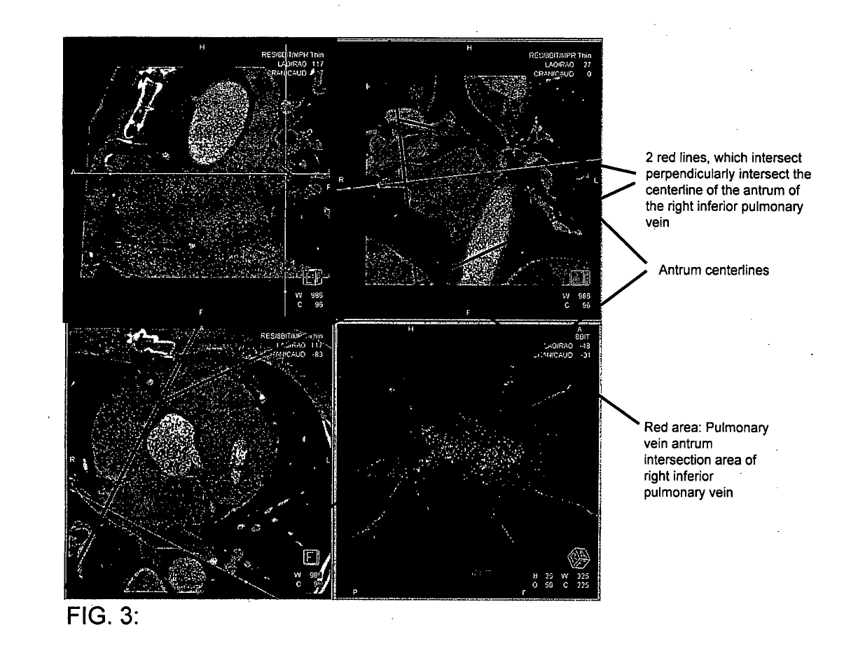

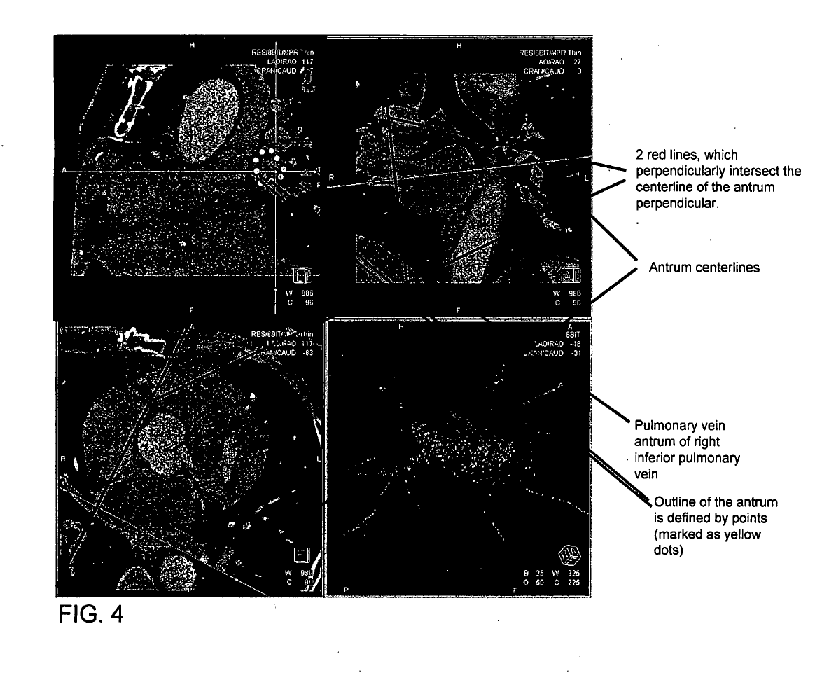

In an example, two of the MPRs may form an orthogonal pair, and a line (shown in FIGS. 3 and 4 ) is aligned so as to intersect the virtual centerline of the pulmonary vein ostium (visible in both of the orthogonal MPRs and shown by a blue line) at a 90 degree angle. This results in an orientation of the third MPR (upper left) such that the antrum is displayed as orthogonal cut. That means the antrum (which may typically be enhanced by contrast agent when the image data is obtained) is displayed in the third MPR as a circular or elliptic shape. That is, the antrum is shown substantially in cross-section. Points identifying the outline of the antrum may be identified by an interactive procedure such as drawing a polygon line or clicking multiple points, or in an automatic manner by 2D segmentation of the antrum in the third MPR, which shows the antrum substantially in cross section. In the figures, the identified points describing the landmark are shown as dots. The identified outline may be shown in a 3D view of the heart, which may be obtained by segmentation of the 3D image data set. A segmented image is displayed in the lower right display segment of FIG. 2.

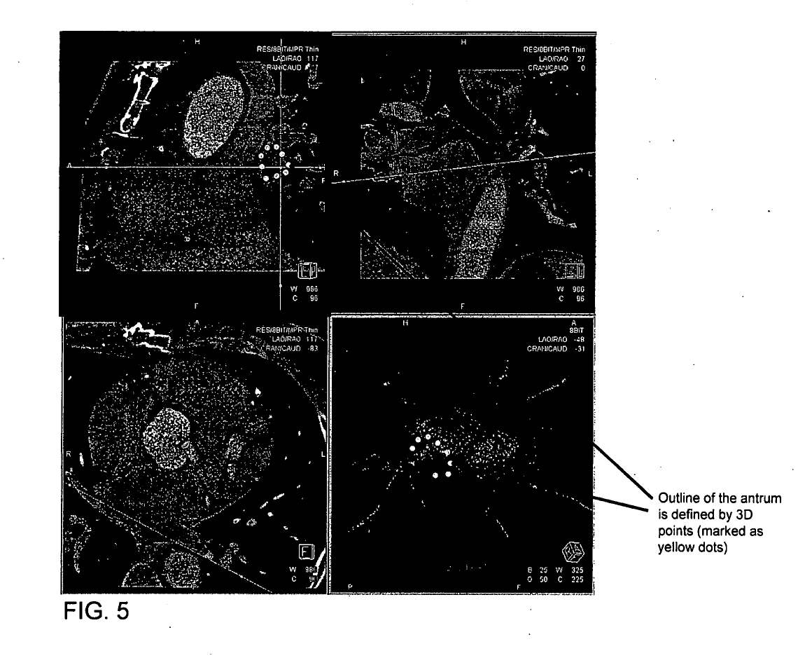

In an alternative to identifying the landmarks within MPRs the landmarks can also be identified in the displayed 3D volume (right lower display segment in FIG. 5). Only one 3D orientation of the segmented organ is shown in FIG. 5, however it should be appreciated that this display is an interactive display and the orientation of the segmented organ may be manipulated by the operator during the process of identifying structures. The MPRs may be caused to rotate correspondingly.

The 3D image display can show the segmented heart chamber as a mesh model or as voxel values. In the later case, the 3D landmark identification may be performed by “3D point picking”. “3D point picking” means that when clicking on the 3D display segment, a surface voxel is selected, which may be defined by the x/y coordinates of the cursor on the displayed image, whereas the z coordinate may be defined by a surface threshold value applied to the voxel data, where the threshold value defines the surface of the 3D object.

In another aspect, the segmented heart chamber may be displayed as a transparent structure. Such a display makes it possible to visualize internal aspects of the organ or structure, such as the pulmonary veins.

In yet another aspect, the spatial contours describing the surface to be ablated (e.g., the interior surface of the segmented left atrium) can be extracted from the 3D display by voxel thresholding. The spatial coordinates of the contour can be transmitted to the X-ray system and can also be displayed on the real-time 2D fluoroscopic images during the procedure. The ablation procedure may also be planned, using electrophysiological data, by marking or transferring coordinates of electrophysiological data onto the displayed images.

The 3D information regarding the identified landmarks, such as the antrum, or the interior surface contours, may be sent from a workstation where the 3D data has been analyzed to the C-arm X-ray system display system over a network. Where the C-arm X-ray system was used to obtain the 3D image data set, the information is already available at the catheter laboratory of FIG. 1. Due to the registration of the 3D and 2D coordinate systems, the landmarks or other graphical information may be merged with and displayed along with the 2D fluoroscopic images.

Whenever the C-arm orientation is changed during the procedure, as may be necessary to facilitate the guidance of an ablation catheter, or to achieve better visibility of a particular structure, the landmarks and any other graphical information is updated with respect to the specific orientation of the C-arm and automatically redrawn so as to be compatible with the image orientation.

By displaying the antrum location landmarks in the real-time 2D fluoroscopic image, the ablation catheter or other treatment device, which is visible in the fluoroscopic image, can be guided relative to the displayed landmark features. Instead of, or in addition to, the antrum outlines a point may be used identify the middle of the antrum of each of the pulmonary veins. Planned ablation lesions can be drawn at the 3D workstation and can be displayed in the 2D fluoroscopic images during the ablation procedure. The 3D spatial features (antrum lines, points identifying the PV ostia, planned ablation lesions) can also be exported to other medical devices used for ablation procedures, such as remote catheter guiding systems (e.g., Niobe from Stereotaxis or Sensei from Hansen Medical) or electroanatomical mapping systems (e.g. CARTO from Biosense Webster or NavX from St. Jude Medical).

In an aspect, a bi-plane X-ray system may be used, so that two orthogonal fluoroscopic images may be obtained simultaneously. In this situation, the 3D landmarks may be visualized in the two 2D images simultaneously.

The extraction and real-time display (in the live 2D fluoroscopic images) of 3D landmarks has been described for atrial fibrillation ablation procedures related to the left atrium. However the method and workflow can be applied also for other electrophysiological procedures or cardiac interventions, wherever real-time display of 3D landmarks may be effective in facilitating the procedure. Other examples of the use of the method may be: marking a heart valve location in valve repair/valve replacement procedures; marking the right atrium and right atrial vessels; using 3D polygon lines for marking cardiac vessels (vessel marking in 3D can be done, for example, by interactive marking in curved MPRs or by automatic centerline extraction) such as coronary veins or coronary arteries; using 3D contours for marking myocardial structures such as hyper-perfused tissue areas, scar areas or areas of limited wall motion or the like; or, using 3D landmarks for marking the foramen ovale in order to support transeptal breakthrough for guiding a catheter from the right atrium into the left atrium. The appropriate organ or structure is segmented using the 3D analysis workstation, and the location of the bodily structure is identified and marked similarly to the atrum as described herein.

A clinical workflow to support the performance of a procedure such as AFib may include the steps of: obtaining 3D image data of the patient using a 3 D imaging modality; analyzing the 3D voxel data to identify one or more landmarks to be used in the procedure; placing the patient in position to perform the procedure; if necessary, registering the 3D coordinate system with the 2D coordinate system to be used intra-procedurally; and, displaying the landmarks on the real-time fluoroscopic images obtained intra-procedurally. The specific procedure to be performed will determine the nature of the landmarks that may be displayed. The landmarks may include points, center lines, transverse planes, surfaces, and the like, projected into the plane of a displayed fluoroscopic image. The fluoroscopic image may also display the radiographic image of any introduced apparatus such as a catheter.

By taking the 3D data set prior to the procedure and using the identified landmarks to mark the real-time fluoroscopic images, the radiation dose to the patient may be reduced, when compared with a situation where 3D images are taken a plurality of times during the procedure.

While the methods disclosed herein have been described and shown with reference to particular steps performed in a particular order, it will be understood that these steps may be combined, sub-divided, or reordered to from an equivalent method without departing from the teachings of the present invention. Accordingly, unless explicitly stated, the order and grouping of steps is not a limitation of the present invention.

Although only a few examples of this invention have been described in detail above, those skilled in the art will readily appreciate that many modifications are possible without materially departing from the novel teachings and advantages of the invention. Accordingly, all such modifications are intended to be included within the scope of this invention as defined in the following claims.

What is claimed is:

1. A system for performing a catheterization procedure, comprising:
a C-arm X-ray device;
a catheter system; and
a computer adapted to:
store a coordinate data set representing a patient bodily structure, the data set obtained by analysis of a three-dimensional (3D) voxel data set;
register the coordinate data set of the bodily structure with respect to a coordinate system of the C-arm X-ray device; and
superimpose a representation of the bodily structure on a real-time fluoroscopic image of the patient obtained by the C-arm X-ray device.

2. The system of claim 1, wherein the catheter system is configurable to perform an electrophysiological (EP) ablation procedure.

3. The system of claim 1, wherein the C-arm X-ray device is used to obtain data for computing the 3D voxel data set.

4. The system of claim 1, wherein the coordinate data set of the bodily structure is determined based on an image data set obtained by a closed computer tomographic (CT) device or a magnetic resonance (MR) imaging device.

5. The system of claim 1, wherein the system further comprises a physiological monitor.

6. The system of claim 5, wherein the physiological monitor is an electrocardiograph (ECG) used to synchronize the image data with a phase of a cardiac cycle of the patient.

7. The system of claim 1, wherein a planned treatment work area is superimposed on the fluoroscopic image.

8. A method of catheter treatment of a patient, the method comprising:
receiving a data set representing a coordinate location of a bodily structure of a patient;
obtaining a fluoroscopic image of the patient;
if necessary, registering the coordinate location with a coordinate system of the fluoroscopic image; and
superimposing the coordinate location of the bodily structure on the fluoroscopic image.

9. The method of claim 8, wherein the coordinate location of a bodily structure is obtained by analysis of a three-dimensional voxel data set of the patient.

10. The method of claim 8, wherein the three dimensional voxel data set is obtained by a computer tomographic device.

11. The method of claim 10, wherein the tomographic device is an X-ray device.

12. The method of claim 10, wherein the tomographic device is a magnetic resonance (MR) imaging device or a closed computed tomographic (CT) device.

13. The method of claim 10 wherein the tomographic device is a C-arm X-ray device.

14. The method of claim 10, wherein the voxel data set is displayed as a plurality of slices.

15. The method of claim 14, wherein two of the slices are orthogonal and an orientation of the third slice is determined by analysis of the orthogonal slices.

16. The method of claim 15, wherein the coordinate location is determined by analysis of the third slice.

17. The method of claim 9, wherein the voxel data set is segmented to display a selected bodily structure.

18. The method of claim 8, further comprising:
providing a catheter system configured to perform an electrophysiological (EP) ablation procedure.

19. A computer program product, the product being stored or distributed on a machine readable medium, comprising:
instructions for causing a computer to perform a method of:
receiving a data set representing a coordinate location of a bodily structure of a patient;
obtaining a fluoroscopic image of the patient;
if necessary, registering the coordinate location with a coordinate system of the fluoroscopic image; and
superimposing the coordinate location of the bodily structure on the fluoroscopic image.
